(19) United States
(12) Patent Application Publication
(10) Pub. No.: US 2010/0028403 A1
(43) Pub. Date: Feb. 4, 2010

(54) MEDICAL DEVICES FOR THERAPEUTIC AGENT DELIVERY

(75) Inventors: Torsten Scheuermann, Munich (DE); Michael Kuehling, Munich (DE)

Correspondence Address: MAYER & WILLIAMS PC, 251 NORTH AVENUE WEST, 2ND FLOOR, WESTFIELD, NJ, 07090, US

(73) Assignee: BOSTON SCIENTIFIC SCIMED, INC., Maple Grove, MN (US)

(21) Appl. No.: 12/511,563

(22) Filed: Jul. 29, 2009

Related U.S. Application Data
(60) Provisional application No. 61/085,169, filed on Jul. 31, 2008.

(51) Int. Cl. A61F 2/00

(52) U.S. Cl. 424/423

(57) ABSTRACT

In various aspects, the present invention relates to implantable or insertable medical devices which release therapeutic agent into the body of a patient.

### RELATED APPLICATIONS

This application claims priority from U.S. provisional application 61/085,169, filed Jul. 31, 2008, which is incorporated by reference herein in its entirety.

### TECHNICAL FIELD

This invention relates to medical devices and, more particularly, to therapeutic-agent-containing medical devices.

### BACKGROUND OF THE INVENTION

The in-situ delivery of therapeutic agents within the body of a patient is common in the practice of modern medicine. In-situ delivery of therapeutic agents is often implemented in conjunction with medical devices that may be temporarily or permanently placed at a target site within the body. These medical devices can be maintained, as required, at their target sites for short or prolonged periods of time, delivering therapeutic agents to the target site.

For example, in recent years, drug eluting coronary stents, which are commercially available from Boston Scientific Corp. (TAXUS), Johnson & Johnson (CYPHER) and others, have become the standard of care for maintaining vessel patency after balloon angioplasty. These existing products are based on metallic expandable stents with polymeric coatings, which release antiproliferative drugs at a controlled rate and total dose.

### SUMMARY OF THE INVENTION

In various aspects, the present invention relates to implantable or insertable medical devices which release therapeutic agent into the body of a patient.

Various aspects, embodiments and advantages of the present invention will become immediately apparent to those of ordinary skill in the art upon review of the Detailed Description and any claims to follow.

### BRIEF DESCRIPTION OF THE DRAWINGS

FIGS. 1-6 are schematic cross sectional illustrations of medical devices in accordance with various embodiments of the invention.

### DETAILED DESCRIPTION

In various aspects, the present invention relates to implantable or insertable medical devices which release therapeutic agent into the body of a patient.

In accordance with certain aspects, medical devices are provided, which comprise a substrate and a barrier layer that at least partially define an enclosed reservoir. The reservoir contains a therapeutic agent and a pressure generating composition, which either actively or passively generates sufficient pressure in vivo to rupture the barrier layer. The pressure generating composition may be, for example, in the form of a liquid or a solid material. In some embodiments, the pressure generating composition may further comprise a therapeutic agent. In some embodiments, a therapeutic agent may be provided within a separate composition (e.g., a separate solid or liquid composition).

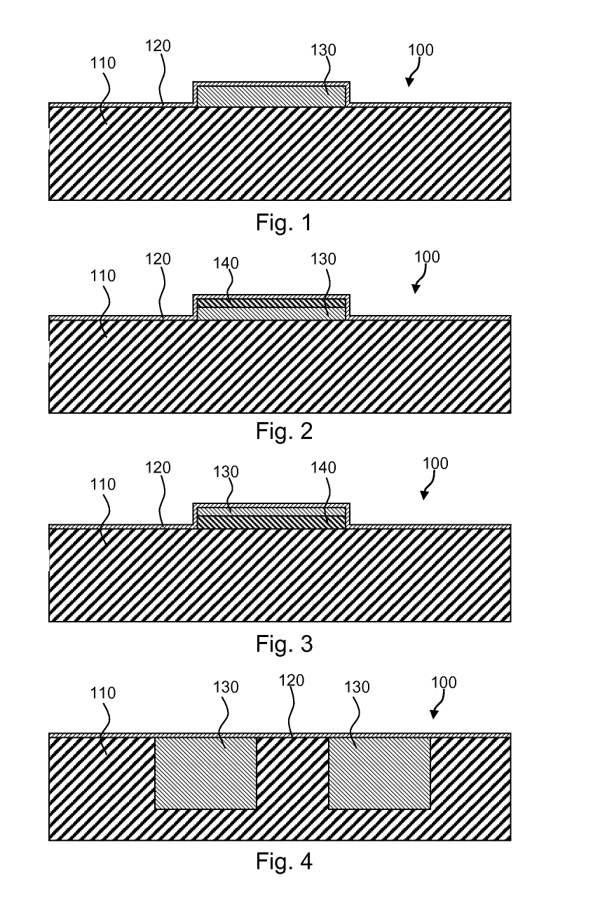

For example, FIG. 1 is a schematic cross-sectional illustration of a medical device 100 in accordance with the invention. The device 100 includes a substrate 1 10. Disposed over the substrate 110 is a pressure generating composition 130. A barrier layer 120 is disposed over the substrate 110 and the pressure generating composition 130. The barrier layer 120 and the substrate 110 cooperate to form an enclosed reservoir for the pressure generating composition 130. As discussed in more detail below, the pressure generating composition 130 is adapted to actively or passively generate sufficient pressure in vivo to rupture the barrier layer 120. Although a single reservoir is shown in FIG. 1, multiple reservoirs can clearly be created by depositing multiple regions of the pressure generating composition 130 on the substrate 110, followed by deposition of a barrier layer 120. In FIG. 1 the pressure generating composition 130 further comprises a therapeutic agent, which is released upon rupture of the barrier layer 120.

FIGS. 2 and 3 are similar to FIG. 1 in that a medical device 100 is shown that includes a substrate 110, a pressure generating composition 130 disposed over the substrate 110, and a barrier layer 120 disposed over the substrate 110 and the pressure generating composition 130, such that the barrier layer 120 and the substrate 110 cooperate to form an enclosed reservoir for the pressure generating composition 130. Unlike FIG. 1, however, a therapeutic agent containing composition 140 is provided, which is distinct from the pressure generating composition 130. Among other possibilities, the therapeutic agent containing composition 140 may be provided in the form of a layer over a layer of the pressure generating composition 130 as shown in FIG. 2 (e.g., for faster release) or, conversely, the pressure generating composition 130 may be provided in the form of a layer over a layer of the therapeutic agent containing composition 140 as shown in FIG. 3 (e.g., for slower release).

In certain embodiments, a first therapeutic agent is contained in the therapeutic agent containing composition 140 and a second therapeutic agent is contained in the pressure generating composition 130, which first and second therapeutic agents may be the same or different. Where the first and second therapeutic agents are the same, the time for release may differ.

In certain embodiments, multiple therapeutic agent containing layers may be provided, in addition to the pressure generating composition. For example, one therapeutic agent containing layer may be provided for faster release and one therapeutic agent containing layer may be provided for slower release of the same or a different therapeutic agent.

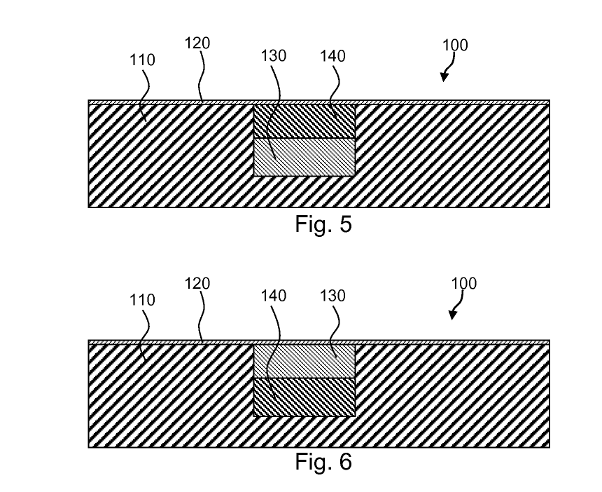

Turning now to FIG. 4, a medical device 100 is shown which includes a substrate 110. Disposed within depressions in the surface of the substrate 110 is a pressure generating composition 130. A barrier layer 120 is disposed over the substrate 110 and the pressure generating composition 130. As above, the barrier layer 120 and the substrate 110 cooperate to form an enclosed reservoir for the pressure generating composition 130. Moreover, the pressure generating composition 130 is adapted to actively or passively generate sufficient pressure in vivo to rupture the barrier layer 120. Although FIG. 4 shows two depressions (and two reservoirs), different number of depressions and reservoirs can clearly be created. In some embodiments, pores within a porous substrate may act as the depressions. In FIG. 4 the pressure generating composition 130 further comprises a therapeutic agent, which is released upon rupture of the barrier layer 120.

FIGS. 5 and 6 are similar to FIG. 4 in that a medical device 100 is shown that includes a substrate 110, a pressure generating composition 130 disposed in a depression in the substrate 110, and a barrier layer 120 disposed over the substrate 110. Unlike FIG. 4, a therapeutic agent containing composition 140 is provided that is distinct from the pressure generating composition 130. Among other possibilities, the therapeutic agent containing composition 140 may be provided in the form of a layer over a layer of the pressure generating composition 130 as shown in FIG. 5 or, conversely, the pressure generating composition 130 may be provided in the form of a layer over a layer of the therapeutic agent containing composition 140 as shown in FIG. 6. A single depression is shown, but clearly, multiple depressions may be employed.

As in FIGS. 2 and 3 above, in certain embodiments, a first therapeutic agent is contained in the therapeutic agent containing composition 140 and a second therapeutic agent is contained in the pressure generating composition 130, which first and second therapeutic agents may be the same or different. Moreover, in certain embodiments, multiple therapeutic agent containing layers may be provided, in addition to the pressure generating composition. For example, one therapeutic agent containing layer may be provided for faster release and one therapeutic agent containing layer may be provided for slower release of the same or a different therapeutic agent.

As indicated above, pressure generating materials include materials that are activated upon implantation with no further effort required of the healthcare practitioner (i.e., passive activation) and materials that can be activated at the command of the healthcare practitioner (i.e., triggered activation).

Examples of the former include materials that swell as a result of the transport of aqueous fluid across the barrier layer from external tissue, including blood, which aqueous fluid is absorbed by the materials, causing to swell. The barrier layer employed may allow for the transport of the aqueous fluid, for example, because it contains one or more openings (e.g., pores, pinholes, etc.) which allow the passage of fluid. In these embodiments, the barrier layer is typically formed of a relatively inelastic material, promoting its rupture under stress. In these embodiments, the pressure generating material may include, for example, a crosslinked hydrophilic polymer (hydrogel) that swells upon exposure to the aqueous fluid, ultimately swelling to the point where the barrier layer is ruptured.

A therapeutic agent may be dispersed within the crosslinked hydrophilic polymer, or a therapeutic agent may be provided in a composition that is distinct from the crosslinked hydrophilic polymer. For example, the therapeutic agent may be provided in a non-aqueous liquid composition or in a solid composition. Examples of non-aqueous liquid compositions include those that comprise the therapeutic agent and one or more organic solvents that do not promote swelling of the crosslinked hydrophilic polymer. Examples of solid compositions include those that comprise the therapeutic agent and a biostable or biodisintegrable polymer (e.g., styrene-isobutylene copolymers, acrylate polymers and copolymers, methacrylate polymers and copolymers, polyesters such as polylactide, polyglycolide and poly(lactide-co-glycolide), etc.), those that comprise the therapeutic agent and a biodisintegrable metallic material (e.g., zinc, iron, magnesium, alloys containing one or more of the same, etc.), those that comprise the therapeutic agent and a biostable metallic or non-metallic inorganic material (e.g., porous metals, porous metal oxides, etc.).

Specific examples of crosslinked hydrophilic materials may be selected from suitable crosslinked homopolymers and copolymers of the following monomers, as well as blends, salts and derivatives of the same, among others: acrylic acid, methacrylic acid, acrylamides such as N-alkylacrylamides, alkylene oxides such as ethylene oxide and propylene oxide, vinyl alcohol, vinylpyrrolidone, vinylpryidines, ethylene imine, ethylene amine, maleic anhydride, acrylonitrile, vinyl sulfonic acid, styrene sulfonate, amino acids such as lysine, histidine, arginine, aspartic acid and glutamic acid. Swellable polymers may further be selected from suitable members of the following: hydrophilic polyurethanes, poly(diallyldimethylammonium chloride), proteins, collagen, cellulosic polymers including methyl cellulose and carboxymethyl cellulose, starch, cationic starch, carboxymethyl starch, dextran, carboxymethyl dextran, modified dextran, alginic acid, pectinic acid, hyaluronic acid, chitin, pullulan, gelatin, gellan, xanthan, albumin, protamine, protamine sulfate, chondroitin sulfate, guar, and blends. The polymers may be covalently crosslinked, non-covalently (e.g., ionically) crosslinked, or both.

Examples of materials that generate pressure in response to external activation include low boiling liquids (e.g., ethanol, acetone, etc.), which may further comprise a therapeutic agent or which may be provided in a composition that is distinct from the therapeutic-agent-containing material (e.g., the therapeutic agent may be may be dispersed in a solid matrix or may be dissolved in a liquid that is immiscible with the vaporizable liquid). The low boiling liquid may be placed, for example, in contact with one or more conductive members that are susceptible to inductive heating. For instance, the conductive members may be in the form of a metallic layer that lines at least a portion of the reservoir that contains the therapeutic agent and pressure generating material. As another example, the conductive members may be in the form of metallic particles that are placed within the reservoir. Upon exposing the conductive members to a magnetic field of suitable frequency and intensity the conductive member heats up (due to the formation of eddy currents in the members), vaporizing the vaporizable liquid. This leads to an increase in pressure in the reservoir, which bursts the membrane. Note that such embodiments allow for therapeutic agent release without the use of microchips or other “smart” electronic devices on the medical device.

Examples of medical devices benefiting from the present invention vary widely and include implantable or insertable medical devices, for example, stents (including coronary vascular stents, peripheral vascular stents, cerebral, urethral, ureteral, biliary, tracheal, gastrointestinal and esophageal stents), catheters (e.g., urological catheters or vascular catheters such as balloon catheters and various central venous catheters), guide wires, balloons, filters (e.g., vena cava filters and mesh filters for distil protection devices), stent coverings, stent grafts, vascular grafts, abdominal aortic aneurysm (AAA) devices (e.g., AAA stents, AAA grafts), vascular access ports, dialysis ports, embolization devices including cerebral aneurysm filler coils (including Guglilmi detachable coils and metal coils), septal defect closure devices, myocardial plugs, patches, pacemakers, leads including pacemaker leads, defibrillation leads, and coils, ventricular assist devices including left ventricular assist hearts and pumps, total artificial hearts, shunts, valves including heart valves and vascular valves, anastomosis clips and rings, cochlear implants, tissue bulking devices, and tissue engineering scaffolds for cartilage, bone, skin and other in vivo tissue regeneration, sutures, suture anchors, tissue staples and ligating clips at surgical sites, cannulae, metal wire ligatures, urethral slings, hernia “meshes”, orthopedic prosthesis such as bone grafts, bone plates, fins and fusion devices, orthopedic fixation devices such as interference screws in the ankle, knee, and hand areas, tacks for ligament attachment and meniscal repair, rods and pins for fracture fixation, screws and plates for craniomaxillofacial repair, artificial ligaments, joint prostheses, dental implants, or other devices that are implanted or inserted into the body and from which therapeutic agent is released or accessed.

Thus, while the devices of the invention in some embodiments may simply provide for release of one or more therapeutic agents as a dosage form, in other embodiments, the medical devices of the invention are configured to provide a therapeutic function beyond therapeutic agent release, for instance, providing mechanical, thermal, magnetic and/or electrical functions within the body, among many other possible functions.

The medical devices of the present invention include, for example, implantable and insertable medical devices that are used for systemic treatment, as well as those that are used for the localized treatment of any mammalian tissue or organ. Non-limiting examples are tumors; organs including the heart, coronary and peripheral vascular system (referred to overall as “the vasculature”), the urogenital system, including kidneys, bladder, urethra, ureters, prostate, vagina, uterus and ovaries, eyes, ears, spine, nervous system, lungs, trachea, esophagus, intestines, stomach, brain, liver and pancreas, skeletal muscle, smooth muscle, breast, dermal tissue, cartilage, tooth and bone.

As used herein, “treatment” refers to the prevention of a disease or condition, the reduction or elimination of symptoms associated with a disease or condition, or the substantial or complete elimination of a disease or condition. Preferred subjects are vertebrate subjects, more preferably mammalian subjects and more preferably human subjects.

Substrate materials for the medical devices of the present invention may vary widely in composition and are not limited to any particular material. They can be selected from a range of biostable materials and biodisintegrable materials (as used herein, “biodisintegrable materials” are materials that are dissolved, degraded, resorbed, or otherwise eliminated upon placement in the body), including (a) organic materials (i.e., materials containing organic species, typically 50 wt % or more) such as polymeric materials (i.e., materials containing polymers, typically 50 wt % or more polymers) and biologics, (b) inorganic materials (i.e., materials containing inorganic species, typically 50 wt % or more), such as metallic materials (i.e., materials containing metals, typically 50 wt % or more) and non-metallic inorganic materials (e.g., including carbon, semiconductors, glasses and ceramics, which may contain various metal- and non-metal-oxides, various metal- and non-metal-nitrides, various metal- and non-metal-carbides, various metal- and non-metal-borides, various metal- and non-metal-phosphates, and various metal- and non-metal-sulfides, among others), and (c) hybrid materials (e.g., hybrid organic-inorganic materials, for instance, polymer/metallic inorganic and polymer/non-metallic inorganic hybrids).

Specific examples of non-metallic inorganic materials may be selected, for example, from materials containing one or more of the following: metal oxides, including aluminum oxides and transition metal oxides (e.g., oxides of titanium, zirconium, hafnium, tantalum, molybdenum, tungsten, rhenium, iron, niobium, and iridium); silicon; silicon-based ceramics, such as those containing silicon nitrides, silicon carbides and silicon oxides (sometimes referred to as glass ceramics); calcium phosphate ceramics (e.g., hydroxyapatite); carbon; and carbon-based, ceramic-like materials such as carbon nitrides.

Specific examples of metallic inorganic materials may be selected, for example, from metals such as gold, silver, iron, nickel, copper, aluminum, niobium, platinum, palladium, iridium, osmium, rhodium, titanium, tantalum, tungsten, ruthenium, zinc and magnesium, among others, and alloys such as those comprising iron and chromium (e.g., stainless steels, including platinum-enriched radiopaque stainless steel), niobium alloys, tantalum alloys, titanium alloys, including alloys comprising nickel and titanium (e.g., Nitinol), alloys comprising cobalt and chromium, including alloys that comprise cobalt, chromium and iron (e.g., elgiloy alloys), alloys comprising nickel, cobalt and chromium (e.g., MP 35N), alloys comprising cobalt, chromium, tungsten and nickel (e.g., L605), alloys comprising nickel and chromium (e.g., inconel alloys), and biodisintegrable alloys including alloys of magnesium, zinc and/or iron (and their alloys with combinations of one another and with Ce, Ca, Zr and Li), among others.

Specific examples of organic materials include a wide variety of biostable and biodisintegrable polymers, along with other high molecular weight organic materials.

As indicated above, in various embodiments of the invention, one or more depressions are formed in the surface of the substrate.

Depressions may be created in various shapes and sizes. Examples include depressions whose lateral dimensions are circular, polygonal (e.g., triangular, quadrilateral, penta-lateral, etc.), as well as depressions of various other regular and irregular shapes and sizes. Multiple depressions can be provided in a near infinite variety of arrays. Examples, of depressions include pores in a porous substrate. Further examples of depressions include trenches, such as simple linear trenches, wavy trenches, trenches formed from linear segments whose direction undergoes an angular change (e.g., zigzag trenches), linear trench networks intersecting various angles, as well as other regular and irregular trench configurations. The depressions can be of any suitable size that provides the features of the invention. For example, the medical devices of the invention typically contain depressions whose smallest lateral dimension (e.g., the width) is less than 10 mm (10000 μm), for example, ranging from 10,000 μm to 1000 μm to 100 μm to 10 μm to 1 μm or less.

Examples of techniques for forming depressions (e.g., pores, holes, trenches, etc.) include methods in which a material contains depressions as-formed. These include molding techniques in which a mold may be provided with various protrusions, which after casting the substrate of interest, create depressions in the material. These techniques further include techniques, such as foam-based techniques, whereby a porous material is formed. Porous materials may also be formed by removing one component from a multi-component material using a suitable process (e.g., dissolution, etching, etc.).

Examples of techniques for forming depressions further include direct removal techniques as well as mask-based removal techniques, in which masking is used to protect material that is not to be removed. Direct removal techniques include those in which material is removed through contact with solid tools (e.g., microdrilling, micromachining, etc.) and those that remove material without the need for solid tools (e.g., those based on directed energetic beams such as laser, electron, and ion beams). Mask-based techniques include those in which the masking material contacts the material to be machined (e.g., where masks are formed using known lithographic techniques) and techniques in which the masking material does not contact the material to be machined, but which is provided between a directed source of excavating energy and the material to be machined (e.g., opaque masks having apertures formed therein, as well as semi-transparent masks such as gray-scale masks which provide variable beam intensity and thus variable machining rates). Material is removed in regions not protected by the above masks using any of a range of processes including physical processes (e.g., thermal sublimation and/or vaporization of the material that is removed), chemical processes (e.g., chemical breakdown and/or reaction of the material that is removed), or a combination of both. Specific examples of removal processes include wet and dry (plasma) etching techniques, and ablation techniques based on directed energetic beams such as electron, ion and laser beams.

Barrier layers for the medical devices of the present invention may vary widely in composition and are not limited to any particular material. They can be selected from a range of biostable materials and biodisintegrable materials, including organic materials (e.g., polymeric materials and biologics) and inorganic materials (e.g., metallic materials and non-metallic inorganic materials). Suitable materials may be selected form those listed above for use as substrate materials. In certain embodiments, the barrier layer is formed of a relatively inelastic material, thereby promoting its rupture under stress. Examples of such materials include low ductility (brittle) polymeric, metallic and non-metallic inorganic materials (e.g., metal oxides, metal nitrides, etc.).

Methods for producing barrier layers include application of a melt or a solution of a barrier material, chemical vapor deposition (CVD), and physical vapor deposition (PVD). Some specific PVD methods that may be used to form barrier layers in accordance with the present invention include evaporation, sublimation, sputter deposition and laser ablation deposition.

As noted above, in some embodiments, a barrier layer is formed, which allows for the transport of the aqueous fluid, because it contains one or more openings (e.g., pores, holes, etc.) which allow the passage of aqueous fluid. Such a barrier layer may be created, for example, by forming one or more holes in the barrier layer using direct removal techniques and mask-based removal techniques methods such as those described above.

Such a barrier may also be created by forming a layer that is porous as-deposited. For example, a porous layer of a biostable or biodisintegrable metallic or non-metallic inorganic material may be deposited using a system available from Mantis Deposition Ltd., Thame, Oxfordshire, United Kingdom, which includes a high-pressure magnetron sputtering source which is able to generate particles from a sputter target with as few as 30 atoms up to those with diameters exceeding 15 nm. A system similar to the Mantis system can be obtained from Oxford Applied Research, Witney, Oxon, UK.

As another example, a porous inorganic oxide barrier layer may be formed using sol-gel techniques.

In some embodiments, a porous barrier layer is formed which comprises first and second materials. Upon implantation, the first material is either reduced in volume or eliminated from the precursor region. For example, a barrier layer may be formed which contains biodisintegrable phase domains (e.g., phase domains of a biodisintegrable metal such as Fe, Mg, Zn, etc.) and biostable metal phase domains (e.g., phase domains of a biostable metal such as Au, Pd, etc.). For example, such a layer may be formed using PVD.

In accordance with the preceding aspects, medical devices are provided, which comprise a substrate and a barrier layer that at least partially define an enclosed reservoir. The reservoir contains a therapeutic agent and a pressure generating composition, which either actively or passively generates sufficient pressure in vivo to rupture the barrier layer.

In other aspects of the invention, medical devices are formed that comprise an enclosed reservoir that contains a therapeutic agent, whereby release of the therapeutic agent from the reservoir into aqueous fluid is increased upon exposure to light, relative to the release that would otherwise occur in the absence of such exposure. Exposure to light may occur in vivo or ex vivo. Typically, the light is ultraviolet (UV) light. In some embodiment, selective irradiation may be used (e.g., using a focused beam or mask) to promote preferential release in some areas of the medical device relative to others.

For example, in some embodiments, the medical device comprises a substrate and a barrier layer, with the substrate and barrier layer at least partially defining the enclosed reservoir that contains the therapeutic agent. Analogous to FIGS. 1 and 4 above, the substrate may or may not contain one or more depressions that define a portion of the enclosed reservoir.

Exposure to light may cause degradation/damage to the barrier layer, for example, leading to breakage of the barrier layer (causing fast release) or leading to increased permeability (but not breakage) of the barrier layer. As a general rule, higher intensity light leads to faster release times. In some embodiments, the increased permeability may lead to increased diffusion of a therapeutic agent across the barrier layer. In some embodiments, the increased permeability may lead to increased diffusion of aqueous fluid into the device, which can result in the generation of pressure within the reservoir, causing breakage of the barrier layer. For example, the reservoir may contain a swellable material such as a crosslinked hydrophilic polymer (hydrogel) or may contain a material (e.g., hydrophilic polymer such as a polysaccharide, polypeptide, etc.) that leads to an increase in osmotic pressure within the reservoir. Analogous to the above, these pressure-generating materials may be admixed with the therapeutic agent (see, e.g., FIGS. 1 and 4) or they may constitute compositions that are distinct from therapeutic-agent-containing compositions (see, e.g., FIGS. 2, 3, 5 and 6).

In some embodiments, degradation/damage to the barrier layer is enhanced through the use of photosensitizers. Examples include aromatic carbonyl photosensitizers (i.e., organic compounds possessing at least one aromatic ring and at least one carbonyl group), for example, aromatic ketones, aromatic diketones, aromatic aldehydes, and aromatic quinones (e.g., substituted and unsubstituted anthraquinone, benzophenone, acetophenone, etc.). Examples further include carboxycyclic diketones and metal complexes thereof. See, e.g., U.S. Pat No. 4,191,320 to Taylor et al. and U.S. Pat. No. 5,274,019 to Poyner et al.

Such photosensitizers may be blended with a polymer to render it more susceptible to UV degradation. Alternatively, a photosensitizer may be incorporated into the polymer chain. Photodegradable polymers can be prepared by copolymerizing a ketone-containing monomer with one or more copolymers. For example, photodegradable polymers may be prepared via addition polymerization or condensation polymerization. For instance, an unsaturated ketone-containing monomer such as an alkyl or aromatic vinyl ketone monomer or an alkyl or aromatic isopropenyl ketone monomer (e.g., methyl vinyl ketone, ethyl vinyl ketone, phenyl vinyl ketone, methyl isopropenyl ketone, ethyl isopropenyl ketone, phenyl isopropenyl ketone etc.) may be addition polymerized with one or more unsaturated comonomers (e.g., alkenes such as ethylene, propylene, isobutylene, alkyl acrylates, alkyl methacryaltes, styrene, etc.). See, e.g., U.S. Pat. No. 3,860,538 to Guillet et al. and the references cited therein. Photodegradable condensation polymers such as polyamides, polyesters, polyurethanes, polyepoxides, polyamide esters, polyureas and polyamino-acids having copolymer backbone of units comprising keto carbonyl groups (e.g., using keto substituted diacids and keto substituted diamines) may also be employed. See, e.g., U.S. Pat. No. 4,042,568 to Guillet et al. and the references cited therein. Photodegradable ethylene-carbon monoxide copolymers may be prepared by peroxide or gamma-ray irradiation initiated copolymerization of ethylene with carbon monoxide, along with optional additional monomers. See, e.g., U.S. Pat. No. 5,219,930 to Chang et al. and the references cited therein. A vinyl alcohol polymer or copolymer (e.g., EVA, etc.) may be rendered photodegradable by reaction with various compounds to provide polymers with pendant carbonyl containing groups, oxycarbonyl containing groups, and keto ether containing groups. See, e.g., U.S. Pat. No. 3,976,621 to Palladino et al., U.S. Pat. No. 5,219,930 to Chang et al., and the references cited therein.

In some embodiments, UV light may be used to render a material in the reservoir more susceptible to swelling. For example, T. Tatsuma et al., Adv. Mater. 2007, 19, 1249-1251 describe an Ag-loaded polyacrylic acid gel which incorporates TiO₂ particles as photocatalysts. When loaded with Ag⁻ the gel is in a shrunken state, reportedly due to electrostatic and coordinative linkages between the carboxyl groups of the polyacrylic acid and the Ag^{|}. When irradiated with UV light in water, the Ag^{|} in the gel is photocatalytically reduced and the gel gradually swells. After the UV light was turned off, the gel continued to swell, gradually slowing down, and then stopped its swelling.

In an embodiment of the invention, such a swellable material may be placed in a reservoir having a water-permeable, UV-transparent barrier layer. A therapeutic agent containing composition may be provided above (assuming that it isn't overly UV absorptive) or below the swellable material in the reservoir. Exposure to UV light in vivo may be used to initiate swelling of the material, ultimately bursting the barrier. Alternatively, exposure UV light ex vivo may be used to initiate swelling, which swelling continues after implantation or insertion of the medical, leading to bursting of the barrier material in vivo.

“Biologically active agents,” “drugs,” “therapeutic agents,” “pharmaceutically active agents,” “pharmaceutically active materials,” and other related terms may be used interchangeably herein and include genetic therapeutic agents, non-genetic therapeutic agents and cells. A wide variety of therapeutic agents can be employed in conjunction with the present invention including those used for the treatment of a wide variety of diseases and conditions (i.e., the prevention of a disease or condition, the reduction or elimination of symptoms associated with a disease or condition, or the substantial or complete elimination of a disease or condition). Numerous therapeutic agents are described here.

Exemplary therapeutic agents for use in conjunction with the present invention include the following: (a) anti-thrombotic agents such as heparin, heparin derivatives, urokinase, clopidogrel, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) alpha receptor antagonist (such as doxazosin, Tamsulosin) and beta receptor agonists (such as dobutamine, salmeterol), beta receptor antagonist (such as atenolol, metaprolol, butoxamine), angiotensin-II receptor antagonists (such as losartan, valsartan, irbesartan, candesartan and telmisartan), and antispasmodic drugs (such as oxybutynin chloride, flavoxate, tolterodine, hyoscyamine sulfate, diclomine) (u) bARKct inhibitors, (v) phospholamban inhibitors, (w) Serca 2 gene/protein, (x) immune response modifiers including aminoquizolines, for instance, imidazoquinolines such as resiquimod and imiquimod, (y) human apolioproteins (e.g., AI, AII, AIII, AIV, AV, etc.), (z) selective estrogen receptor modulators (SERMs) such as raloxifene, lasofoxifene, arzoxifene, miproxifene, ospemifene, PKS 3741, MF 101 and SR 16234, (aa) PPAR agonists, including PPAR-alpha, gamma and delta agonists, such as rosiglitazone, pioglitazone, netoglitazone, fenofibrate, bexaotene, metaglidasen, rivoglitazone and tesaglitazar, (bb) prostaglandin E agonists, including PGE2 agonists, such as alprostadil or ONO 8815Ly, (cc) thrombin receptor activating peptide (TRAP), (dd) vasopeptidase inhibitors including benazepril, fosinopril, lisinopril, quinapril, ramipril, imidapril, delapril, moexipril and spirapril, (ee) thymosin beta 4, (ff) phospholipids including phosphorylcholine, phosphatidylinositol and phosphatidylcholine, (gg) VLA-4 antagonists and VCAM-1 antagonists.

Numerous therapeutic agents, not necessarily exclusive of those listed above, have been identified as candidates for vascular treatment regimens, for example, as agents targeting restenosis (antirestenotic agents). Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, such as bosentan, sitaxsentan sodium, atrasentan, endonentan, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) Angiotensin Converting Enzyme (ACE) inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, atorvastatin, fluvastatin, simvastatin and cerivastatin, (u) fish oils and omega-3-fatty acids, (v) free-radical scavengers/antioxidants such as probucol, vitamins C and E, ebselen, trans-retinoic acid, SOD (orgotein), SOD mimics, verteporfin, rostaporfin, AGI 1067 and M 40419, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) matrix metalloprotease (MMP) pathway inhibitors such as marimastat, ilomastat metastat, batimastat, pentosan polysulfate, rebimastat, incyclinide, apratastat, PG 116800, RO 1130830 or ABT 518, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate , nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), olimus family drugs (e.g., sirolimus, everolimus, tacrolimus, zotarolimus, etc.), cerivastatin, flavopiridol and suramin, (aa) matrix deposition/organization pathway inhibitors such as halofuginone or other quinazolinone derivatives, pirfenidone and tranilast, (bb) endothelialization facilitators such as VEGF and RGD peptide, (cc) blood rheology modulators such as pentoxifylline, and (dd) glucose cross-link breakers such as alagebrium chloride (ALT-711).

Preferred therapeutic agents in some embodiments include taxanes such as paclitaxel (including particulate forms thereof, for instance, protein-bound paclitaxel particles such as albumin-bound paclitaxel nanoparticles, e.g., ABRAXANE), sirolimus, everolimus, tacrolimus, zotarolimus, Epo D, dexamethasone, estradiol, halofuginone, cilostazole, geldanamycin, alagebrium chloride (ALT-711), ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, imiquimod, human apolioproteins (e.g., AI-AV), growth factors (e.g., VEGF-2), as well derivatives of the foregoing, among others.

A wide range of therapeutic agent loadings may be used in conjunction with the medical devices of the present invention. Typical loadings for a given therapeutic agent containing composition may range, for example, from than 1 wt % or less to 2 wt % to 5 wt % to 10 wt % to 25 wt % or more of the composition.

Numerous additional therapeutic agents useful for the practice of the present invention are also disclosed in U.S. Pat. No. 5,733,925 to Kunz et al.

Although various embodiments are specifically illustrated and described herein, it will be appreciated that modifications and variations of the present invention are covered by the above teachings and are within the purview of the appended claims without departing from the spirit and intended scope of the invention.

1. A medical device comprising a substrate and a barrier layer, said substrate and said barrier layer at least partially defining an enclosed reservoir, said medical device further comprising a therapeutic agent and a crosslinked hydrophilic polymer disposed within the reservoir, wherein the reservoir is permeable to water such that, when the medical device is implanted in a patient, aqueous fluid enters the reservoir and swells the crosslinked hydrophilic polymer to a point where the barrier is burst.

2. The medical device of claim 1, wherein said substrate comprises a depression which defines at least a portion of said reservoir.

3. The medical device of claim 1, wherein said crosslinked hydrophilic polymer is selected from polyacrylic acid, polyethylene oxide, polyvinyl alcohol and gelatin.

4. The medical device of claim 1, wherein said barrier layer is an inorganic barrier layer.

5. The medical device of claim 1, wherein said barrier layer is permeable to aqueous fluid.

6. The medical device of claim 1, wherein the therapeutic agent is dispersed within the crosslinked hydrophilic polymer.

7. The medical device of claim 1, wherein the therapeutic agent is provided in a composition that is distinct from the crosslinked hydrophilic polymer.

8. The medical device of claim 1, wherein the medical device is selected from a stent, a stent graft, a pacemaker electrode, a neurostimulation implant, an infusion pump, a vascular access device, and an orthopedic implant.

9. A medical device comprising an enclosed reservoir that contains a therapeutic agent, wherein a release of the therapeutic agent from the reservoir into surrounding aqueous fluid is increased upon exposure to light, relative to the release that would otherwise occur in the absence of said exposure.

10. The medical device of claim 9, wherein said medical device comprises a substrate and a barrier layer, and wherein said substrate and said barrier layer at least partially define said enclosed reservoir.

11. The medical device of claim 10, wherein said substrate comprises a depression which at least partially defines said enclosed reservoir.

12. The medical device of claim 10, wherein said light is UV light.

13. The medical device of claim 12, wherein said barrier layer is a UV degradable barrier layer.

14. The medical device of claim 12, wherein said barrier layer is a UV degradable polymeric layer.

15. The medical device of claim 14, wherein said UV degradable polymeric layer comprises a photosensitizer.

16. The medical device of claim 12, wherein said barrier layer is transmissive to UV light and permeable to aqueous fluid, and wherein said reservoir comprises a polymer gel that is rendered susceptible to swelling in aqueous fluid upon exposure to UV light.

17. A medical device comprising a substrate and a barrier layer, said substrate and said barrier layer at least partially defining an enclosed reservoir, said medical device further comprising a therapeutic agent and a vaporizable liquid within the reservoir, wherein the vaporizable liquid is adjacent to a conductive material that can be heated to vaporize the vaporizable liquid and break the barrier layer by subjecting the conductive material to a magnetic field of suitable frequency and intensity.

18. The medical device of claim 17, wherein said substrate comprises a depression which defines at least a portion of said reservoir.

19. The medical device of claim 17, wherein the vaporizable liquid is selected from ethanol and acetone.
